(19) United States
(12) Patent Application Publication
(10) Pub. No.: US 2019/0336024 A1
(43) Pub. Date: Nov. 7, 2019

(54) BRAIN-BASED THOUGHT IDENTIFIER AND CLASSIFIER

(71) Applicant: INTERNATIONAL BUSINESS MACHINES CORPORATION, ARMONK, NY (US)

(72) Inventors: Nicolo' Sgobba, Fasano (IT); Erik Rueger, Ockenheim (DE); Athila Mattos, Brno (CZ); Sergi Takahashi, Brno (CZ); Eugen Salnikov, Brno (CZ)

(21) Appl. No.: 15/972,312

(22) Filed: May 7, 2018

(51) Int. Cl. A61B 5/04; A61B 5/0476; G06N 3/08; A61B 5/00

(52) U.S. Cl. CPC A61B 5/04012; A61B 5/0476; G06N 3/08; G06F 3/015; A61B 5/7257; A61B 5/7225; A61B 5/7267

(57) ABSTRACT

Embodiments provide thought classifier devices that convert analog electroencephalogram signals obtained from mental activity of a person to a digital signal bitstream data; identify a portion of the bitstream as a thought chunk representing discrete thought activity in response to matching, via a first artificial neural network comparison, digital signal bitstream thought chunk portion metadata to metadata labeled in association with a thought within a thoughts data set, the first artificial neural network trained on the thoughts data set; identify a user category in response to matching, via a different, second artificial neural network comparison, metadata of the thought chunk portion to labeled metadata within the thoughts data set, the second artificial neural network trained on the thoughts data set; and identify a specific thought of the thoughts data that has metadata that has corresponding metadata.

### BACKGROUND

Brain Computer Interfaces (BCI) refers to processing devices that attempt to monitor, identify or determine animal brain activity from electrical activity, or images generated therefrom, etc., and use the monitored activity to function as control inputs for motorized or a computerized device. BCI's commonly observe or acquire electroencephalogram (EEG) signals to represent brain activity, such as by recording electrical activity of the brain obtainable from sensors applied externally to the scalp of a person, wherein the wave forms recorded thereby are interpreted to reflect electrical activity of proximate regions of the brain, and more specifically, of the cortex. Conventional analogical EEG measuring instruments include an amplifier, a galvanometer and a writing device. Digital EEG systems convert or transform acquired analog EEG waveforms into numerical value representations via analog-to-digital conversion (ADC) processes. Thus converted, the digital representations of the analog waveforms can be processed, manipulated and operated upon through a variety of mathematical processes to generate new data or make various determinations, as well as stored into a computer memory.

### SUMMARY

In one aspect of the present invention, a computerized method for a thought classifier includes executing steps on a computer processor. Thus, computer processors are configured to convert analog electroencephalogram signals obtained from mental activity of a person to a digital signal bitstream data. The configured processors identify a portion of the digital signal bitstream data as a thought chunk that represents discrete thought activity in response to matching, via a first artificial neural network comparison, first metadata of the thought chunk portion of the digital signal bitstream to metadata that is labeled in association with a thought within a thoughts data set, wherein the first artificial neural network is trained on the thoughts data set. The configured processors identify a user category to which the person generating the mental activity belongs in response to matching, via a second artificial neural network comparison, the first metadata and length and generation time metadata of the thought chunk portion to metadata that is labeled in association with a user category within the thoughts data set, wherein the second artificial neural network is trained on the thoughts data set and is different from the first artificial neural network. Thus, the configured processors identify a specific thought within the thoughts data that has metadata that corresponds to the first metadata, the length and generation time metadata of the thought chunk portion, and to metadata that is labeled in association with the identified user category within the thoughts data set.

In another aspect, a system has a hardware processor in circuit communication with a computer readable memory and a computer-readable storage medium having program instructions stored thereon. The processor executes the program instructions stored on the computer-readable storage medium via the computer readable memory and is thereby configured to convert analog electroencephalogram signals obtained from mental activity of a person to a digital signal bitstream data. The configured processor identifies a portion of the digital signal bitstream data as a thought chunk that represents discrete thought activity in response to matching, via a first artificial neural network comparison, first metadata of the thought chunk portion of the digital signal bitstream to metadata that is labeled in association with a thought within a thoughts data set, wherein the first artificial neural network is trained on the thoughts data set. The configured processor identifies a user category to which the person generating the mental activity belongs in response to matching, via a second artificial neural network comparison, the first metadata and length and generation time metadata of the thought chunk portion to metadata that is labeled in association with a user category within the thoughts data set, wherein the second artificial neural network is trained on the thoughts data set and is different from the first artificial neural network. Thus, the configured processor identifies a specific thought within the thoughts data that has metadata that corresponds to the first metadata, the length and generation time metadata of the thought chunk portion, and to metadata that is labeled in association with the identified user category within the thoughts data set.

In another aspect, a computer program product for a thought classifier has a computer-readable storage medium with computer readable program code embodied therewith. The computer readable hardware medium is not a transitory signal per se. The computer readable program code includes instructions for execution which cause the processor to convert analog electroencephalogram signals obtained from mental activity of a person to a digital signal bitstream data. The processor is caused to identify a portion of the digital signal bitstream data as a thought chunk that represents discrete thought activity in response to matching, via a first artificial neural network comparison, first metadata of the thought chunk portion of the digital signal bitstream to metadata that is labeled in association with a thought within a thoughts data set, wherein the first artificial neural network is trained on the thoughts data set. The processor is caused to identify a user category to which the person generating the mental activity belongs in response to matching, via a second artificial neural network comparison, the first metadata and length and generation time metadata of the thought chunk portion to metadata that is labeled in association with a user category within the thoughts data set, wherein the second artificial neural network is trained on the thoughts data set and is different from the first artificial neural network. Thus, the processor is caused to identify a specific thought within the thoughts data that has metadata that corresponds to the first metadata, the length and generation time metadata of the thought chunk portion, and to metadata that is labeled in association with the identified user category within the thoughts data set.

### BRIEF DESCRIPTION OF THE DRAWINGS

These and other features of embodiments of the present invention will be more readily understood from the following detailed description of the various aspects of the invention taken in conjunction with the accompanying drawings in which:

FIG. 1 depicts a cloud computing environment according to an embodiment of the present invention.

FIG. 2 depicts abstraction model layers according to an embodiment of the present invention.

FIG. 3 depicts a computerized aspect according to an embodiment of the present invention.

FIG. 4 is a flow chart illustration of an embodiment of the present invention.

FIG. 5 is a flow chart illustration of another embodiment of the present invention.

FIG. 6 is a graphical illustration of a bitstream chunk identification according to an implantation of the present invention.

FIG. 7 is a flow chart illustration of another embodiment of the present invention.

FIG. 8 is a flow chart illustration of another embodiment of the present invention.

### DETAILED DESCRIPTION

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

It is to be understood that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and be rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported, providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure that includes a network of interconnected nodes.

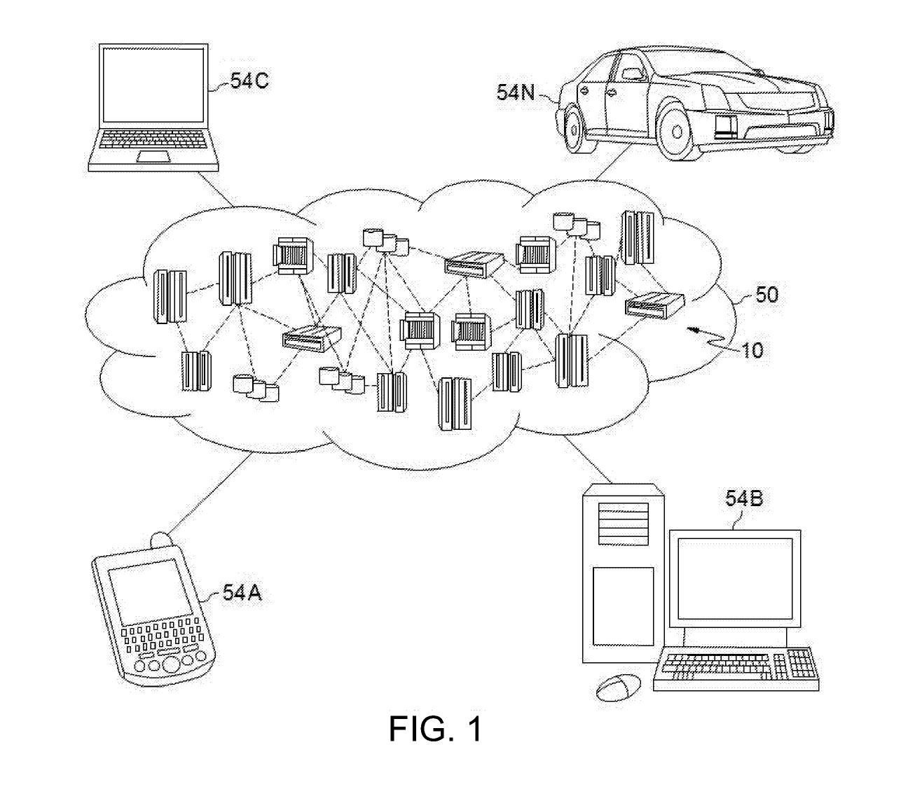

Referring now to FIG. 1, illustrative cloud computing environment 50 is depicted. As shown, cloud computing environment 50 includes one or more cloud computing nodes 10 with which local computing devices used by cloud consumers, such as, for example, personal digital assistant (PDA) or cellular telephone 54A, desktop computer 54B, laptop computer 54C, and/or automobile computer system 54N may communicate. Nodes 10 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 1 are intended to be illustrative only and that computing nodes 10 and cloud computing environment 50 can communicate with any type of computerized device over any type of network and/or network addressable connection (e.g., using a web browser).

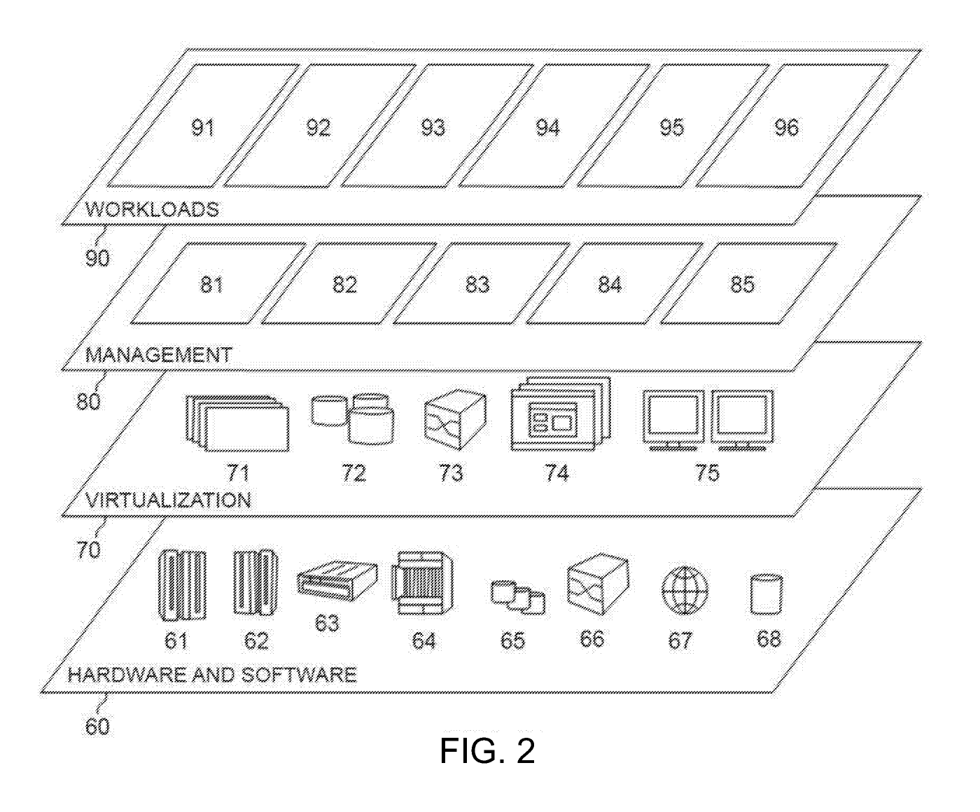

Referring now to FIG. 2, a set of functional abstraction layers provided by cloud computing environment 50 (FIG. 1) is shown. It should be understood in advance that the components, layers, and functions shown in FIG. 2 are intended to be illustrative only and embodiments of the invention are not limited thereto. As depicted, the following layers and corresponding functions are provided:

Hardware and software layer 60 includes hardware and software components. Examples of hardware components include: mainframes 61; RISC (Reduced Instruction Set Computer) architecture based servers 62; servers 63; blade servers 64; storage devices 65; and networks and networking components 66. In some embodiments, software components include network application server software 67 and database software 68.

Virtualization layer 70 provides an abstraction layer from which the following examples of virtual entities may be provided: virtual servers 71; virtual storage 72; virtual networks 73, including virtual private networks; virtual applications and operating systems 74; and virtual clients 75.

In one example, management layer 80 may provide the functions described below. Resource provisioning 81 provides dynamic procurement of computing resources and other resources that are utilized to perform tasks within the cloud computing environment. Metering and Pricing 82 provide cost tracking as resources are utilized within the cloud computing environment, and billing or invoicing for consumption of these resources. In one example, these resources may include application software licenses. Security provides identity verification for cloud consumers and tasks, as well as protection for data and other resources. User portal 83 provides access to the cloud computing environment for consumers and system administrators. Service level management 84 provides cloud computing resource allocation and management such that required service levels are met. Service Level Agreement (SLA) planning and fulfillment 85 provide pre-arrangement for, and procurement of, cloud computing resources for which a future requirement is anticipated in accordance with an SLA.

Workloads layer 90 provides examples of functionality for which the cloud computing environment may be utilized. Examples of workloads and functions which may be provided from this layer include: mapping and navigation 91; software development and lifecycle management 92; virtual classroom education delivery 93; data analytics processing 94; transaction processing 95; and processing for a thought classifier according to embodiments of the present invention 96.

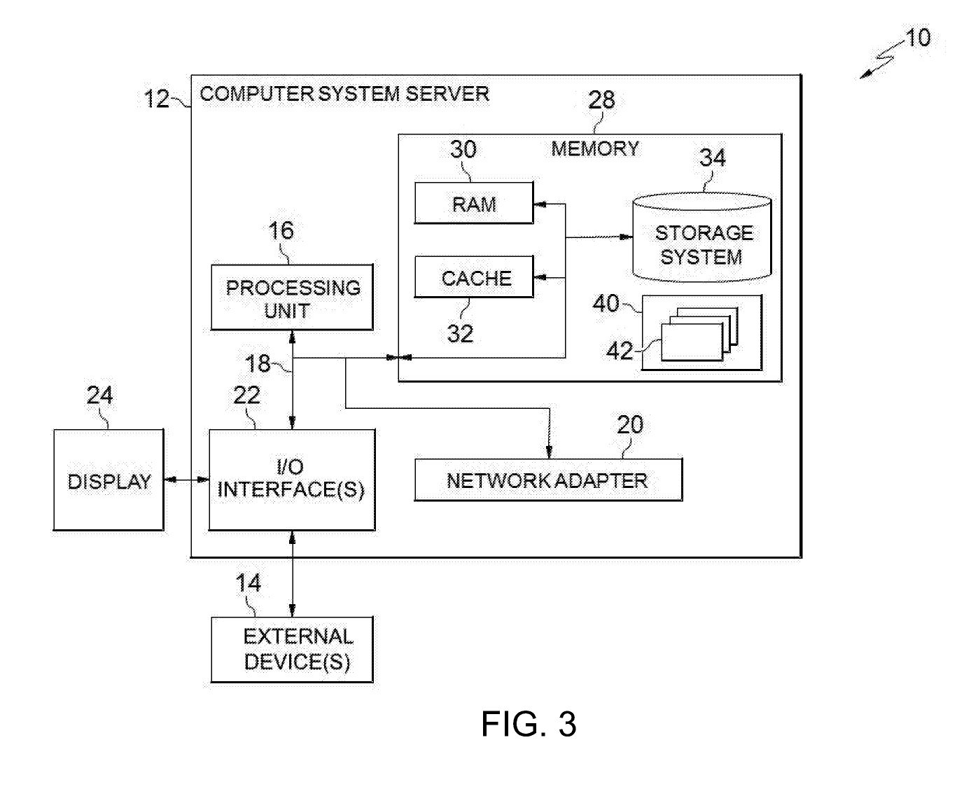

FIG. 3 is a schematic of an example of a programmable device implementation 10 according to an aspect of the present invention, which may function as a cloud computing node within the cloud computing environment of FIG. 2. Programmable device implementation 10 is only one example of a suitable implementation and is not intended to suggest any limitation as to the scope of use or functionality of embodiments of the invention described herein. Regardless, programmable device implementation 10 is capable of being implemented and/or performing any of the functionality set forth hereinabove.

A computer system/server 12 is operational with numerous other general purpose or special purpose computing system environments or configurations. Examples of well-known computing systems, environments, and/or configurations that may be suitable for use with computer system/server 12 include, but are not limited to, personal computer systems, server computer systems, thin clients, thick clients, hand-held or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

Computer system/server 12 may be described in the general context of computer system-executable instructions, such as program modules, being executed by a computer system. Generally, program modules may include routines, programs, objects, components, logic, data structures, and so on that perform particular tasks or implement particular abstract data types. Computer system/server 12 may be practiced in distributed cloud computing environments where tasks are performed by remote processing devices that are linked through a communications network. In a distributed cloud computing environment, program modules may be located in both local and remote computer system storage media including memory storage devices.

The computer system/server 12 is shown in the form of a general-purpose computing device. The components of computer system/server 12 may include, but are not limited to, one or more processors or processing units 16, a system memory 28, and a bus 18 that couples various system components including system memory 28 to processor 16.

Bus 18 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnects (PCI) bus.

Computer system/server 12 typically includes a variety of computer system readable media. Such media may be any available media that is accessible by computer system/server 12, and it includes both volatile and non-volatile media, removable and non-removable media.

System memory 28 can include computer system readable media in the form of volatile memory, such as random access memory (RAM) 30 and/or cache memory 32. Computer system/server 12 may further include other removable/non-removable, volatile/non-volatile computer system storage media. By way of example only, storage system 34 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 18 by one or more data media interfaces. As will be further depicted and described below, memory 28 may include at least one program product having a set (e.g., at least one) of program modules that are configured to carry out the functions of embodiments of the invention.

Program/utility 40, having a set (at least one) of program modules 42, may be stored in memory 28 by way of example, and not limitation, as well as an operating system, one or more application programs, other program modules, and program data. Each of the operating system, one or more application programs, other program modules, and program data or some combination thereof, may include an implementation of a networking environment. Program modules 42 generally carry out the functions and/or methodologies of embodiments of the invention as described herein.

Computer system/server 12 may also communicate with one or more external devices 14 such as a keyboard, a pointing device, a display 24, etc.; one or more devices that enable a user to interact with computer system/server 12; and/or any devices (e.g., network card, modem, etc.) that enable computer system/server 12 to communicate with one or more other computing devices. Such communication can occur via Input/Output (I/O) interfaces 22. Still yet, computer system/server 12 can communicate with one or more networks such as a local area network (LAN), a general wide area network (WAN), and/or a public network (e.g., the Internet) via network adapter 20. As depicted, network adapter 20 communicates with the other components of computer system/server 12 via bus 18. It should be understood that although not shown, other hardware and/or software components could be used in conjunction with computer system/server 12. Examples, include, but are not limited to: microcode, device drivers, redundant processing units, external disk drive arrays, RAID systems, tape drives, and data archival storage systems, etc.

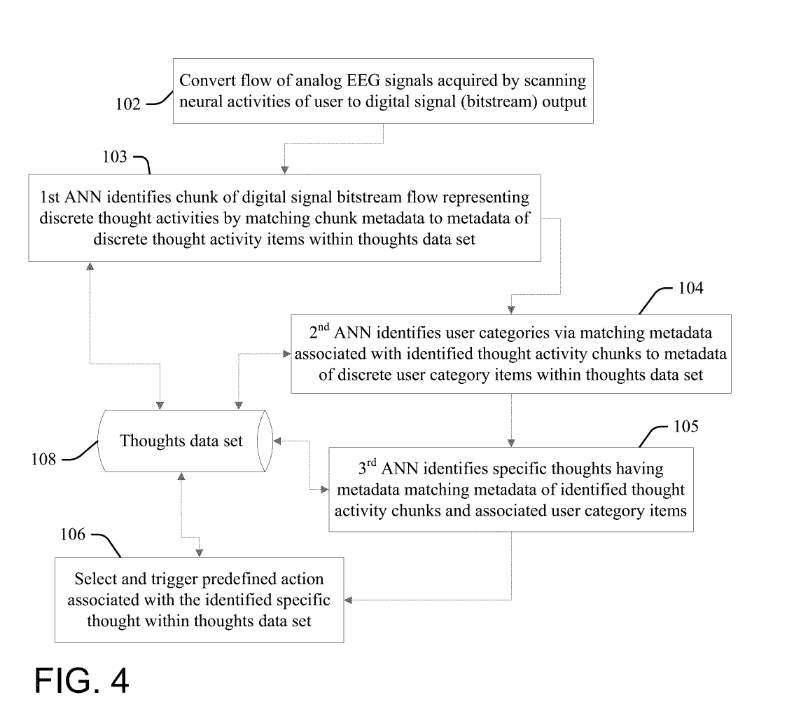

FIG. 4 illustrates a thought classifier according to embodiments of the present invention. A processor (for example, a central processing unit (CPU)) executes code (such as code installed on a storage device in communication with the processor) and is thereby configured according to the present invention (a “configured processor”) to function as a neural sensors interface (NSI) 102, wherein at 102 the configured processor acquires a continuous flow of EEG signal inputs from a user by scanning current neural activities of the user while the user generates thoughts, and converts the analog EEG signals to a digital signal (bitstream) output that it passes to a first trained artificial neural network (ANN) that defines a chunk identifier (CI) 103.

An ANN comprises a plurality of model “neuron” weighting elements that exchange messages between each other via numerically-weighted decision connections that are tuned (weighted) to correspond to neural processes of a user (a person thinking that is scanned to provide EEG signal inputs) represented by digitized EEG training set data. An ANN model weight set is trained into weighting factors at by setting weightings to correspond to digitized EEG data profile patterns that are distinguished and replicated within the user's EEG data acquired from thoughts and activities of users, including when a user goes through repetitive iterations of an associated mental activity (a particular muscle movement, an image or command visualization, etc.). Training the weighting factors causes them to correspond to digitized EEG data profile pattern distinguished and replicated within the training set of digitized user EEG data.

Thus, a first ANN chunk identifier at 103 comprises a processor configured according to the present invention (“configured processor”) that identifies within the digital signal bitstream flow a portion or “chunk” of the digital signal bitstream that represents discrete thought activity, by matching metadata associated with and/or derived from the portion of the digital bitstream to metadata labeled in association with a thought within a thoughts data set or database 108 that is used to train the first ANN chunk identifier 103; and passes the identified thought activity chunk to another, second trained ANN, a user classifier (UC), at 104. The metadata values or features considered for matching at 103 include metadata of the analog electroencephalogram signals that are converted into the thought chunk portion of the digital signal bitstream: illustrative but not limiting or exhaustive examples of said analog signal metadata include frequency values of the (raw) analog EEG signals converted to the bitstream data at 102; fast-Fourier transform (FFT) values of the frequency values of the analog EEG signals; amplitude values of the analog EEG signals; FFT values of the amplitude values of the analog EEG signals; voltage values of the analog EEG signals; and signal length values of the analog EEG signals. Still other metadata values appropriate for use in the metadata matching process at 103 will be apparent to one skilled in the art.

The fast-Fourier transform generically transforms a signal from the time domain into the frequency domain, wherein the time-dependent signal analog signal data is broken down into a collection of sinusoids, wherein lengthy and noisy EEG analog signal data is transformed and plotted in a frequency power-spectrum. Thus, metadata and temporal information inherently contained within the original analog data is generated by the ADC process, and this additional feature data is used by the first ANN at 103 to improving training and accuracy in the process of identifying the chunks.

At 104 a second ANN configured as a user classifier (UC) comprises a processor configured according to the present invention (“configured processor”) that identifies a user category to which the user generating the thoughts (the person generating the mental activity) belongs, via matching metadata associated with the identified thought activity chunk to a metadata of a discrete user category item within a data set of the thoughts data set 108 that is used to train the ANN user classifier 104; and passes the identified thought activity chunk in associated with the identified user category item to another, third trained ANN: a chunk classifier (CC) 105. In one example, training data set is generated by user-defined examples to train the ANN user classifier 104 via a supervised machine learning approach, wherein the goal of the training phase is to train the system to separate the data into different groups or classes, and wherein errors from an initial classification of initial (first) records are fed back into the neural network and thereby used to modify underlying algorithm-based processes during second and subsequent iterations, generally for many iterations. The defined model is then used to classify or identify single chunks with respect to user category items based on data classification of the individual metadata.

At 105 a third ANN chunk classifier is configured as a chunk thought classifier (CC) and comprises a processor configured according to the present invention (“configured processor”) that identifies a specific thought within a data set of the thoughts data set 108 used to train the 3rd ANN chunk classifier that has metadata that corresponds to (matches) the metadata of the identified thought activity chunk and the metadata of the identified user category associated to the identified chunk at 104.

At 106 a processor configured according to the present invention (“configured processor”) selects and triggers a predefined action that is associated with the specific thought identified at 105 within the thoughts data set 108. Thus, the present embodiment triggers or execute an action identified as fulfilling the user's thought or intent as determined from the EEG analog inputs, providing a generic thoughts identification system that identifies a user's thought and executes an appropriate, predefined action based on said thought identification. According to the capabilities, characteristics and the available computing resources of the embodiments of the present invention, the component 106 is capable of triggering or executing steps required to fulfill the identified thought: for example, if the identified thought is “turn off outer door vestibule lights,” at 106 the embodiment may autonomously send a packets of data to an “Internet-of-Things” (IoT) lighting device located within an outer door vestibule of a residence of a user generating the identified thought 606 (via a networked communication structure) that causes the IoT light to turn off if on, or verify that the light is off.

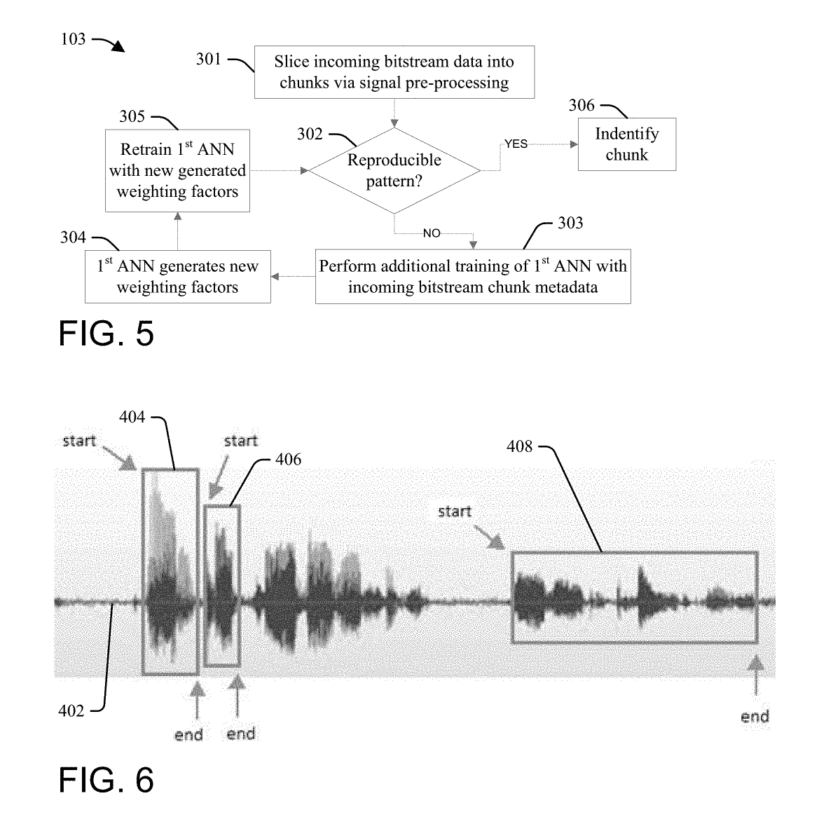

Performance of each of the 1^{st} ANN CI 103, the 2^{nd} ANN UC 104 and the 3^{rd} ANN CC 105 is improved by using pre-defined training data set examples, that are defined and structured within the thoughts database 108 according to an appropriate data model, both during initial training and during iterative retraining as discussed with respect to FIGS. 5, 7 and 8 below.

Embodiments of the thoughts database 108 are generally properly dimensioned to store hundreds of thousands of examples of tuples that link (include) each of identified thoughts, the related and most relevant metadata values (features) considered by each of the 1^{st} ANN CI 103, the 2^{nd} ANN UC 104 and the 3^{rd} ANN CC 105, and the actions to be executed by the action component 106. For example, a tuple may comprise string data of an identified thought (“apple”, “light switch” or “computer mouse pointer”), an identified bitstream chunk (“10101010110”), a length value of the identified bitstream chunk (“8”), a user category (“student”), a level of education of the user (“3^{rd}-year undergraduate”), expertise of the user (“student of electrical engineering”), school attended (“Big State University”), generation time of the raw EEG signals converted to the chunk bitstream (“1.2 seconds”), voltage of the raw EEG signals (“5 millivolts (mV)”), predefined action (“render image of apple on graphical user display,” “change light switch position from current state” or “move pointer”).

Embodiments of the present invention may also define a data lifecycle for a data model, for example, in order to describe a data persistence from a conception time through a time of disposal. Data may be created and entered into the thoughts database 108 as manual input for additional data annotation, to define the example training dataset, as well as during a training phase including user feedback, and still other models will be apparent to one skilled in the art. Accordingly, in some embodiments the database 108 is no longer required for the system to properly operate when the Neural Networks that compose the first, second and third ANN's system are determined to be fully trained and capable of providing correct cognitive decisions, wherein the thoughts database 108 is disposed of or otherwise disconnected or not used in the classification processes at 103, 104 and/or 105 once training is complete.

FIG. 5 illustrates one embodiment of the first ANN chunk-identifier process at 103. The bitstream incoming (input) from 102 with its set of metadata values (features) is processed at 302, so that a cognitive decision is performed: identify possible chunks of the bitstream as a single thought activity chunks 306 in response to determining that the frequency domain patterns of the chunk are reproducible (“yes” condition). Otherwise, in response to determining at 302 that the frequency domain patterns of the chunk are not reproducible (“no” condition), the embodiment performs additional training of the 1^{st} ANN with the non-reproducible chunk at 303, generates new weighting factors at 304 and retrains the ANN with the new generated weighting factors at 305, for iterative reconsideration of the chunk at 302, until the chunk is determined to be reproducible at 302 and accordingly identified as a chunk at 306. Thus, the process steps 302-303-304-305 provide further training of the 1^{st} ANN, to enable more accurate decisions at 302. Accuracy may also be improved at 302 by additional neural network training phase iterations with appropriate training datasets.

Pre-processing at 301 may comprise chunking the incoming bitstream based on meeting threshold signal variations values indicative of abrupt changes in thought activity by the user. Significant changes in relative values of the bitstream metadata over time are more likely to be associated with changes to new and discrete thoughts in the user mental activity, and possibly in a correspondingly higher probability of a correct chunk classification.

FIG. 6 illustrates one example of a “sliding window” chunking process at 301, an approach for signal processing of a that considers “windows” 404, 406 and 408 defined by threshold changes in frequency domain and/or metadata values of the bitstream data flow 402 considered over time (as defined by their respective “start” and “stop” times along the horizontal (x-axis) time values. The windows 404, 406 and 408 may “slide” across a time series portion of the signal so that, given a fixed time frame (for example, 3 seconds) and a start point in time identified as having meeting a threshold variation in frequency value relative to the value at a previous time, the window can be extended as needed based on an identified end point (identified as being, again, having a significant, threshold signal variation with respect to a value at a subsequent time). Window start-point and end-point identification may also be based on changes in associated, respective metadata values at said time points. Thus, the portion of the signal delimited between the start and the end points of the respective windows 404, 406 and 408 are potential chunks identified at 301 within the incoming bitstream flow that are classified as chunks at 306 in response to determining at 302 that they are reproducible.

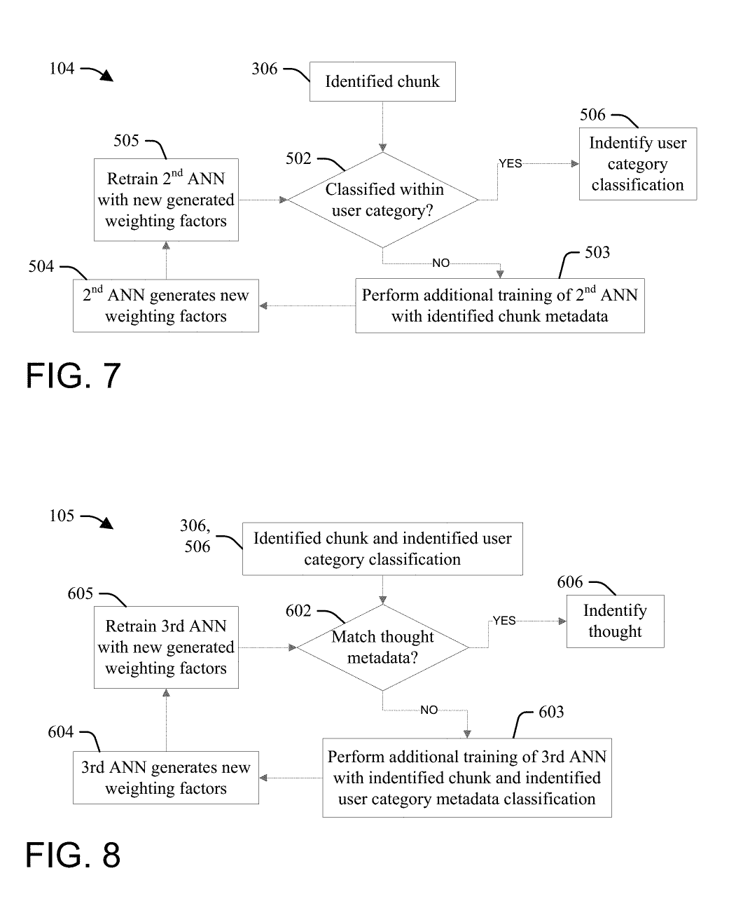

FIG. 7 illustrates one embodiment of the second ANN user classifier at 104. A single chunk identified (at FIG. 5 306) by the 1^{st} ANN CI 103 represents a single thought, wherein a cognitive decision process at 502 determines whether a user category of the chunk 306 is identifiable as a function of related and most relevant metadata values (features) of the identified chunk. Illustrative but not limiting or exhaustive examples of the identified chunk 306 metadata values considered at 502 include chunk length metrics (for example, total number of “one” and “zeros” defining the identified chunk 306; total generation time of the identified chunk 306, and the metadata considered at 302 of FIG. 5 (frequency values of the (original, raw) analog EEG wave signals of the identified chunk 306 that were converted to the bitstream data at 102; fast-Fourier transform (FFT) values of said frequency values of the analog EEG signals; amplitude values of the (original, raw) analog EEG signals of the identified chunk 306; FFT values of said amplitude values of the analog EEG signals; voltage values of the (original, raw) analog EEG signals of the identified chunk 306; and signal length values of the analog EEG signals). Still other metadata values appropriate for processing and comparison in the user category identification process at 502 will be apparent to one skilled in the art.

Thus, if the identified chunk 306 is determined to be classifiable as to user category at 502 (“yes” condition), the user category is identified at 506. Otherwise, in response to determining at 502 that a user category is not identifiable (“no” condition), the embodiment performs additional training of the second ANN user classifier (104) with the identified chunk at 503, generates new weighting factors for the 2^{nd} ANN at 504 and retrains the 2^{nd} ANN with the new generated weighting factors at 505, for iterative reclassification of the identified chunk at 502, until the identified chunk is classified within a user category at 506. Thus, the process steps 502-503-504-505 provide further training of the 2^{nd} ANN, to enable more accurate user category classifications at 502, and wherein accuracy may be improved at 502 by additional neural network training phase iterations with appropriate training datasets.

FIG. 8 illustrates one embodiment of the third ANN user classifier at 105. The identified chunk 306 and its user category identified at 506 and their related and relevant metadata values are input to a cognitive decision process at 602 which determines whether their metadata values match metadata values associated with a specific, distinct thought within a thought dataset (thus, within the thoughts dataset 108 of FIG. 4). Illustrative but not limiting or exhaustive examples of the metadata values of the identified chunk 306 and identified user category 506 considered at 602 include user demographic data (illustrative but not limiting or exhaustive examples include occupation, student status, education certification and attainments, and still other demographic categories of the user appropriate for practicing with embodiments of the present invention and will be apparent to one skilled in the art), the identified chunk 306 length metrics; the total generation time of the identified chunk 306, the frequency values of the (original, raw) analog EEG wave signals of the identified chunk 306 that were converted to the bitstream data at 102; the fast-Fourier transform (FFT) values of said frequency values of the analog EEG signals; the amplitude values of the (original, raw) analog EEG signals of the identified chunk 306; the FFT values of said amplitude values of the analog EEG signals; the voltage values of the (original, raw) analog EEG signals of the identified chunk 306; and the signal length values of the analog EEG signals. Still other metadata values appropriate for processing and comparison in the thought identification process at 602 will be apparent to one skilled in the art.

Thus, if the identified chunk 306 and identified user category 506 metadata is determined at 602 to match or otherwise be classifiable as to the metadata of the user thought within a database of thoughts 108 user category (“yes” condition), the matching/classified thought is identified at 606. Otherwise, in response to determining at 602 that the identified chunk 306 and identified user category 506 metadata does not match or otherwise may not be classifiable as to the metadata of a user thought within the database of thoughts 108 (“no” condition), the embodiment performs additional training of the third ANN user classifier (105) with the identified chunk and the identified user category 506 metadata at 603, generates new weighting factors for the 3rd ANN at 604 and retrains the 3rd ANN with the new generated weighting factors at 605, for iterative matching classifications to thoughts within the thought database at 602, until the identified chunk 306 and identified user category 506 metadata is matched with an identifiable thought at 606. Thus, the process steps 602-603-604-605 provide further training of the 3rd ANN, to enable more accurate though identifications at 602, and wherein accuracy may be improved at 602 by additional neural network training phase iterations with appropriate training datasets.

Generating training thoughts and associate metadata for the thought database 108, or the determinations at 302, 502 or 602, may comprise assuring reproducibility of digital representations of the EEG activity signals and/or their analog metadata via iteratively testing, for example, by prompting the user to repeat a current activity and again generate EEG signal data mapped to the ANN elements and weighted by learned weights until a number of weighted transformations of the EEG data obtained from each iteration match with a threshold degree of similarity and frequency (for example, over 50%, 66.7%, 75%, 90% of three or more repetitions). In some aspects the assessment directly informs the user supplying the current EEG signals (for example, visually via a graphical user interface (GUI) display device, and/or acoustically through audio instructions), in a real-time feedback process, as to whether the current EEG signals have a sufficient reproducibility and distinctiveness qualities to be used in combination with other, different and distinct weighted EEG signal sets provided by the user to generate a valid personal signature. Upon verification that generation of weighted EEG signal sets is repeatable by the user, embodiments may verify the uniqueness of the associated ANN weight set is verified: for example, that the weighting values distinguish thoughts and user categories are computationally complex enough that they cannot be replicated by (mistaken for) another. Thus, a single ANN weight set may be used to define a metadata set of a thought chunk that is unique to a thought and user category, though some embodiments use combinations or patterns of multiple, different ANN weight sets that are each associated with a different thought or user category to define the reproducible EEG metadata signatures.

Embodiments may also define thought datasets or ANN weightings at a variety of granularities. For example, different respective activities used to generate weighted sets of EEG metadata may include EEG signal data generated in response to directing muscle movements or the absence thereof, such as user eyeball movements, specific hand movements and patterns (such as to trace out or write an alphanumeric character), and other sequential combinations of a variety of different movements, as well as signals generated from “no activity” or “pauses” in movement wherein the user holds him or herself still and makes no volitional muscle movements for a “pause” period of time. The different respective activities may also include different mental thoughts, exercises or processes, including memorizing or recalling a specific image or picture, different alpha-numeric characters. Thus, embodiments of the present invention may use ANN model estimates or approximations of neural functions of the user that are represented by the EEG inputs as a system (“net”) of interconnected components (“neurons”) that exchange messages between each other and have numeric weights that are tuned to generate a unique set of weight values that function as unique thought representations for the user and/or user category.

Embodiments may be easily and efficiently deployed via wearable EEG sensors that can be integrated into a hat, baseball cap, spectacles and sunglasses, headbands, earphones, hair clips, ear rings, etc., that enable EEG electrodes to be placed upon or close enough to the user's head surface to detect EEG signals, and which may be transferred securely via encrypted Bluetooth®, Wi-Fi or other wireless network connections to a user's smart phone or other portable, programmable device. (BLUETOOTH is a trademark of Bluetooth SIG in the United States or other countries.) Because the EEG signals are very low frequency they can also be modulated onto a higher digital carrier frequency, including via unique ANN model weightings trained for each person, enabling the transfer of captured EEG signals to another computer device in an optimal quality via an encrypted network connection.

Embodiments may generate training thought metadata in response to (labeled in association with) specific “presentment activity,” sensory stimulus with respect to an identifiable item, sensation, etc., that triggers mental activity by the user to identify the item/ sensation. Thus, a presentment activity training period or phase may obtain EEG signals while the person is asked to process an image (for example, to identify a tree, table, person or other object, or to identify an activity depicted in the image (a person running, smiling, etc.), complete a math problem, identify a missing item on a picture, etc.; to identify auditory data (a spoken word or number, or number of different notes or tones presented via an audio speaker to the person); to describe a sensation (for example, a pulsing pressure on a finger, a heat sensation, etc.); and still other examples will be apparent to one skilled in the art; to thereby acquire EEG signals while the person is actively engaged in mental activity needed to perform the identification, or counting or memorization and recall, etc. task. Thus, the training set EEG signal profile portions obtained from other persons during corresponding presentment activity training periods are labeled as “presentment activity” signal portions within the training set compared by the configured processor to the signals obtained, to identify those portions that match the training set portions labeled as presentment activity, and to responsively label these identified portions as “presentment activity” signal portions; and to further adjust (increase) the weight of the matching signal portions labeled as presentment activity within ANN training set data more heavily relative to the other signal portions within the training set labeled as presentment activity that do not match EEG signal portion profiles obtained from a user.

Embodiments may inherently identify common profile portions within EEG results as a function of a variety of different characteristics and categories of the sensory data presented that result in identifiable EEG signal portions that are common or similar across persons within certain user categories used to train the ANN. For example, if the stimulation provided to a person of a first user category (for example, a 3^{rd}-year liberal arts undergraduate with no prior degree) is a picture of a red car of a particular make, model or type, the EEG signals may be generated in response to the person thinking about the car (object identification), the color (property), shape (feature), brand (characteristic), etc. Where the embodiment trains the ANN's on EEG signals obtained from a plurality of presentations to different persons within the first user category, the EEG result profile portions are each may be labeled or flagged with the first user category. This increases the chances of a positive match at 302, 502 or 602, because when the embodiment system presents the same image to another user, the response provided will be matched not just with the results of other cars, but also with other results having the same color, brand, feature, etc. Differences between EEG signals generated from car images that differ as to color, brand, feature, etc., will be automatically and autonomously be determined by the 1^{st}, 2^{nd} or 3^{rd} ANN's, as represented in corresponding similarities or differences in the respective weightings of labeled EEG signal portions executed by the respective ANN, based on similarities or differences in color, shape, object type or category, etc., with respect to presented stimuli.

The terminology used herein is for describing aspects only and is not intended to be limiting of the invention. As used herein, the singular forms “a”, “an” and “the” are intended to include the plural forms as well, unless the context clearly indicates otherwise. It will be further understood that the terms “include” and “including” when used in this specification specify the presence of stated features, integers, steps, operations, elements, and/or components, but do not preclude the presence or addition of one or more other features, integers, steps, operations, elements, components, and/or groups thereof. Certain examples and elements described in the present specification, including in the claims, and as illustrated in the figures, may be distinguished, or otherwise identified from others by unique adjectives (e.g. a “first” element distinguished from another “second” or “third” of a plurality of elements, a “primary” distinguished from a “secondary” one or “another” item, etc.) Such identifying adjectives are generally used to reduce confusion or uncertainty, and are not to be construed to limit the claims to any specific illustrated element or embodiment, or to imply any precedence, ordering or ranking of any claim elements, limitations, or process steps.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A computer-implemented method for a thought classifier, the method comprising executing on a computer processor:
converting analog electroencephalogram signals to a digital signal bitstream data, wherein the analog electroencephalogram signals are obtained from mental activity of a person;
identifying a portion of the digital signal bitstream data as a thought chunk that represents discrete thought activity in response to matching, via a first artificial neural network comparison, first metadata of the thought chunk portion of the digital signal bitstream to metadata that is labeled in association with a thought within a thoughts data set, wherein the first artificial neural network is trained on the thoughts data set;
identifying a user category to which the person generating the mental activity belongs in response to matching, via a second artificial neural network comparison, the first metadata and length and generation time metadata of the thought chunk portion to metadata that is labeled in association with a user category within the thoughts data set, wherein the second artificial neural network is trained on the thoughts data set and is different from the first artificial neural network; and
identifying a specific thought within the thoughts data that has metadata that corresponds to the first metadata, the length and generation time metadata of the thought chunk portion, and to metadata that is labeled in association with the identified user category within the thoughts data set.

2. The method of claim 1, further comprising:
integrating computer-readable program code into a computer system comprising a processor, a computer readable memory in circuit communication with the processor, and a computer readable storage medium in circuit communication with the processor; and
wherein the processor executes program code instructions stored on the computer-readable storage medium via the computer readable memory and thereby performs the converting the analog electroencephalogram signals to the digital signal bitstream data, the identifying the portion of the digital signal bitstream data as the thought chunk in response to the matching via the first artificial neural network comparison, the identifying the user category in response to the matching via the second artificial neural network comparison, and the identifying the specific thought within the thoughts.

3. The method of claim 2, wherein the computer-readable program code is provided as a service in a cloud environment.

4. The method of claim 1, wherein the identifying the specific thought comprises:
matching, via a third artificial neural network comparison, the first metadata, the length and generation time metadata of the thought chunk portion and the metadata that is labeled in association with the identified user category to metadata that is labeled in association with the specific thought within the thoughts data set, wherein the third artificial neural network is trained on the thoughts data set and is different from the first artificial neural network and from the second artificial neural network.

5. The method of claim 4, wherein the first metadata of the thought chunk portion comprises metadata of the analog electroencephalogram signals that are converted into the thought chunk portion of the digital signal bitstream that is selected from the group consisting of frequency values of the analog electroencephalogram signals, fast-Fourier transform values of the frequency values, amplitude values of the analog electroencephalogram signals, fast-Fourier transform values of the amplitude values, voltage values of the analog electroencephalogram signals, and signal length values of the analog electroencephalogram signals.

6. The method of claim 5, further comprising:
triggering a predefined action that is associated with the specific thought within the thoughts data set.

7. The method of claim 6, wherein association of the predefined action with the specific thought within the thoughts data set is defined by a tuple that comprises a string data representation of the specific thought, the portion of the digital signal bitstream data identified as the thought chunk, a length value of the portion of the digital signal bitstream data identified as the thought chunk, a string data representation of the identified user category, string data descriptive of a demographic attribute of the person, and string data descriptive of the predefined action.

8. The method of claim 7, further comprising identifying the portion of the digital signal bitstream data as the thought chunk as a window portion of the analog electroencephalogram signals converted to the digital signal bitstream data digital signal bitstream data that is defined by a threshold changes in frequency domain values of the analog electroencephalogram signals over time at beginning and ending times of the window portion.

9. The method of claim 7, wherein the string data descriptive of a demographic attribute of the person is selected from the group consisting of a level of education of the person, a level of expertise of the person, and a school attended by the person.

10. A system, comprising:
a processor;
a computer readable memory in circuit communication with the processor; and
a computer readable storage medium in circuit communication with the processor;
wherein the processor executes program instructions stored on the computer-readable storage medium via the computer readable memory and thereby:
converts analog electroencephalogram signals to a digital signal bitstream data, wherein the analog electroencephalogram signals are obtained from mental activity of a person;
identifies a portion of the digital signal bitstream data as a thought chunk that represents discrete thought activity in response to matching, via a first artificial neural network comparison, first metadata of the thought chunk portion of the digital signal bitstream to metadata that is labeled in association with a thought within a thoughts data set, wherein the first artificial neural network is trained on the thoughts data set;
identifies a user category to which the person generating the mental activity belongs in response to matching, via a second artificial neural network comparison, the first metadata and length and generation time metadata of the thought chunk portion to metadata that is labeled in association with a user category within the thoughts data set, wherein the second artificial neural network is trained on the thoughts data set and is different from the first artificial neural network; and
identifies a specific thought within the thoughts data that has metadata that corresponds to the first metadata, the length and generation time metadata of the thought chunk portion, and to metadata that is labeled in association with the identified user category within the thoughts data set.

11. The system of claim 10, wherein the processor executes the program instructions stored on the computer-readable storage medium via the computer readable memory and thereby identifies the specific thought in response to matching, via a third artificial neural network comparison, the first metadata, the length and generation time metadata of the thought chunk portion and the metadata that is labeled in association with the identified user category to metadata that is labeled in association with the specific thought within the thoughts data set, wherein the third artificial neural network is trained on the thoughts data set and is different from the first artificial neural network and from the second artificial neural network.

12. The system of claim 11, wherein the first metadata of the thought chunk portion comprises metadata of the analog electroencephalogram signals that are converted into the thought chunk portion of the digital signal bitstream that is selected from the group consisting of frequency values of the analog electroencephalogram signals, fast-Fourier transform values of the frequency values, amplitude values of the analog electroencephalogram signals, fast-Fourier transform values of the amplitude values, voltage values of the analog electroencephalogram signals, and signal length values of the analog electroencephalogram signals.

13. The system of claim 12, wherein the processor executes the program instructions stored on the computer-readable storage medium via the computer readable memory and thereby triggers a predefined action that is associated with the specific thought within the thoughts data set.

14. The system of claim 13, wherein association of the predefined action with the specific thought within the thoughts data set is defined by a tuple that comprises a string data representation of the specific thought, the portion of the digital signal bitstream data identified as the thought chunk, a length value of the portion of the digital signal bitstream data identified as the thought chunk, a string data representation of the identified user category, string data descriptive of a demographic attribute of the person, and string data descriptive of the predefined action.

15. The system of claim 14, wherein the processor executes the program instructions stored on the computer-readable storage medium via the computer readable memory and thereby identifies the portion of the digital signal bitstream data as the thought chunk as a window portion of the analog electroencephalogram signals converted to the digital signal bitstream data digital signal bitstream data that is defined by a threshold changes in frequency domain values of the analog electroencephalogram signals over time at beginning and ending times of the window portion.

16. A computer program product for a thought classifier, the computer program product comprising:
a computer readable storage medium having computer readable program code embodied therewith, wherein the computer readable storage medium is not a transitory signal per se, the computer readable program code comprising instructions for execution by a processor that cause the processor to:
convert analog electroencephalogram signals to a digital signal bitstream data, wherein the analog electroencephalogram signals are obtained from mental activity of a person;
identify a portion of the digital signal bitstream data as a thought chunk that represents discrete thought activity in response to matching, via a first artificial neural network comparison, first metadata of the thought chunk portion of the digital signal bitstream to metadata that is labeled in association with a thought within a thoughts data set, wherein the first artificial neural network is trained on the thoughts data set;
identify a user category to which the person generating the mental activity belongs in response to matching, via a second artificial neural network comparison, the first metadata and length and generation time metadata of the thought chunk portion to metadata that is labeled in association with a user category within the thoughts data set, wherein the second artificial neural network is trained on the thoughts data set and is different from the first artificial neural network; and
identify a specific thought within the thoughts data that has metadata that corresponds to the first metadata, the length and generation time metadata of the thought chunk portion, and to metadata that is labeled in association with the identified user category within the thoughts data set.

17. The computer program product of claim 16, wherein the computer readable program code instructions for execution by the processor further cause the processor to identify the specific thought in response to matching, via a third artificial neural network comparison, the first metadata, the length and generation time metadata of the thought chunk portion and the metadata that is labeled in association with the identified user category to metadata that is labeled in association with the specific thought within the thoughts data set, wherein the third artificial neural network is trained on the thoughts data set and is different from the first artificial neural network and from the second artificial neural network.

18. The computer program product of claim 17, wherein the first metadata of the thought chunk portion comprises metadata of the analog electroencephalogram signals that are converted into the thought chunk portion of the digital signal bitstream that is selected from the group consisting of frequency values of the analog electroencephalogram signals, fast-Fourier transform values of the frequency values, amplitude values of the analog electroencephalogram signals, fast-Fourier transform values of the amplitude values, voltage values of the analog electroencephalogram signals, and signal length values of the analog electroencephalogram signals.

19. The computer program product of claim 18, wherein the computer readable program code instructions for execution by the processor further cause the processor to trigger a predefined action that is associated with the specific thought within the thoughts data set.

20. The computer program product of claim 19, wherein association of the predefined action with the specific thought within the thoughts data set is defined by a tuple that comprises a string data representation of the specific thought, the portion of the digital signal bitstream data identified as the thought chunk, a length value of the portion of the digital signal bitstream data identified as the thought chunk, a string data representation of the identified user category, string data descriptive of a demographic attribute of the person, and string data descriptive of the predefined action.
